(19) United States
(12) Patent Application Publication
(10) Pub. No.: US 2024/0225841 A1
(43) Pub. Date: Jul. 11, 2024

(54) X-Ray Developable Identifying Element for Use in an Implant and Implant Comprising the Same

(71) Applicant: New J company, Taipei (TW)

(72) Inventor: Cheng-Han HSIEH, Taipei (TW)

(21) Appl. No.: 18/583,919

(22) Filed: Feb. 22, 2024

Related U.S. Application Data
(63) Continuation of application No. PCT/CN2022/078742, filed on Mar. 2, 2022.

(51) Int. Cl. A61F 2/28; A61F 2/16; A61F 2/18

(52) U.S. Cl. CPC A61F 2/28; A61F 2/16; A61F 2/186; A61F 2002/2835; A61F 2250/0086

(57) ABSTRACT

Provided is an X-Ray developable identifying element for use in an implant, which comprises a body and an identifying pattern; wherein the identifying pattern is formed on the body or embedded in the body, and the material of the identifying pattern is an X-ray developable material. Provided is an implant comprising the X-Ray developable identifying element. The identifying element has the X-ray developable identifying pattern, so the image of identifying pattern will be displayed on an X-ray film after X-ray radiation to represent the origin of the product and model number, which allows the patients or users to identify the information of the implant implanted in their bodies. The present disclosure allows patients or users to obtain the information of the implant implanted in their bodies outside the body so as to improve the convenience of getting the information of the implant implanted in patients or users.

### CROSS REFERENCE

This application is a continuation of PCT Patent Application PCT/CN2022/078742, filed on Mar. 2, 2022. The content of the above application is incorporated herein by its entirety.

### BACKGROUND OF THE INVENTION

### 1. Field of the Invention

The present invention relates to an implant comprising an identifying pattern, and particularly to an implant which can provide its information after implanted into the human body.

### 2. Description of the Prior Arts

With the advancement of medicine and medical materials, the use of implants in human bodies has become increasingly common. Implants such as pacemakers, heart stents, breast implants, artificial nose implants, artificial bone, artificial blood vessels, intraocular lenses, dental bone grafts, serve various needs and enhance the convenience of life and confidence of patients or users. Additionally, aesthetic medicine and reconstruction involving the implantation of medical devices in pursuit of beauty is also prevalent. Due to the fact that implants typically remain in the body for a long period of time after surgical implantation, the quality of implants has always been a concern for patients or users. Furthermore, according to the Department of Medical Affair of Taiwan's Ministry of Health and Welfare's draft amendments to the Physicians Act issued in June 2020, physicians are required to record the information of brand, model, and lot number of any implant that will remain in the body for more than 30 days in the patient's medical records for tracking.

However, implant-related information such as brand, model, lot number, material, place of origin, and year of manufacture is only recorded in the medical records in the original medical institutions in the current state; therefore, when patients or users seek medical treatment or consultation at different medical institutions other than the one where the implant was initially implanted, the lack of the patient's or user's previous medical records or information about the implant in the latter medical institution makes it difficult for the physician to timely access information about the implant in the patient's or user's body. They often need to access the patient's or user's medical records at the original medical institution to obtain information about the implant. Additionally, if patients or users wish to inquire about the information related to the implant in their bodies, they can only return to the original medical institution where the implantation was performed, causing inconvenience in obtaining the information.

### SUMMARY OF THE INVENTION

In light of the drawback of not able to timely obtain the information of the implant in the human body, the present invention provides an X-ray developable identifying element for use in an implant which can make the product information accessible outside the human body, so that the information of the implant inside human body can be accessed easily for the medical personnel, patients, or users.

To achieve the above purpose, the present invention provides an X-ray developable identifying element for use in an implant, comprising a body and an identifying pattern; wherein the identifying pattern is formed on the body or embedded in the body, and the material of the identifying pattern is an X-ray developable material. Because the X-ray developable identifying element has the identifying pattern that can develop after X-ray radiation, the image of the identifying pattern can be displayed on an X-ray film when the identifying pattern is used in an implant implanted in the human body. This allows for the display of information such as the brand and model of the implant, and enables patients or users to obtain the relevant information about the implant inside their bodies.

According to the present invention, the X-ray developable material refers to a material which can develop an image on an e X-ray film after X-ray radiation. Preferably, the X-ray developable material may comprise an X-Ray developer or a metal. Preferably, the X-Ray developer may be a barium sulfate developer or an iodine-containing developer.

Preferably, the identifying pattern comprises a two-dimensional barcode. Preferably, the two-dimensional barcode may be a quick-response code, data matrix code, or Aztec code. The two-dimensional barcode can be read by a reader to further provide more detailed information of the implant, such as lot number, material, place of origin, and so on.

Preferably, the identifying pattern is formed on the body or embedded in the body by printing or laser.

Preferably, the body can be in the form of different shapes, including, but not limited to, a shape of a disc, a sphere, or a capsule.

Preferably, the “identifying pattern embedded in the body” refers to that the identifying pattern is formed on a substrate embedded in the body.

Preferably, the material of the body and the substrate is an X-Ray undevelopable material. Preferably, the X-Ray undevelopable material is a non-metallic material. Preferably, the non-metallic material is ceramic, glass, polyurethane or silicone. In one embodiment, the non-metallic material is silicone.

In one embodiment, a flange is formed outward from the upper end of the body, so that the body is attached to the implant with the flange when the X-ray developable identifying element is used in an implant.

To achieve the above purpose, the present invention further provides an implant, comprising a carrier, and the aforementioned X-ray developable identifying element; wherein the X-Ray developable identifying element is disposed on or embedded in the carrier.

Preferably, the material of the carrier is an X-Ray undevelopable material. Preferably, the X-Ray undevelopable material is a non-metallic material. Preferably, the non-metallic material is ceramic, glass, polyurethane or silicone. In one embodiment, the non-metallic material is silicone.

Preferably, the carrier comprises a filler and an out-layer, wherein a surface of the filler is covered by the out-layer; when the X-Ray developable identifying element is disposed on the carrier, the X-Ray developable identifying element is disposed on the out-layer; and when the X-Ray developable identifying element is embedded in the carrier, the X-ray developable identifying element is embedded in the filler.

Preferably, the X-ray developable identifying element is disposed on the out-layer, and the identifying pattern on the X-ray developable identifying element faces to the filler, so that the body rejection hardly occurs when the implant is implanted in a human body.

Preferably, the material of the filler and/or the out-layer is an X-ray undevelopable material. For example, the material of the filler and/or the out-layer is silicone. Preferably, an X-ray undevelopable material is a silicone gel. Preferably, the material of the out-layer is silicone. In one embodiment, the identifying element is in the shape of a disc, and the diameter thereof is 0.4 centimeter (cm) to 1 cm. More preferably, the diameter thereof is 0.4 cm to 0.8 cm.

For example, the implant is a breast implant, an artificial nose implant, an artificial bone, an artificial blood vessel, an intraocular lens or a dental bone graft.

According to the present invention, the carrier may be in the form of different shapes, including, but not limited to, a shape of an arc, a disc, a cone, an irregular-shape, a square cone, and a tubular shape.

In one embodiment, the X-ray developable identifying element is embedded in and secured in the carrier. In another embodiment, the X-ray developable identifying element is embedded in the carrier and the X-ray developable identifying element is movably disposed in the carrier.

By using the technical means described above, the X-ray developable identifying element of the present invention and the implant comprising the same have the identifying pattern, so that the product information of the implant implanted in the human body, such as the brand, model, lot number, material, origin, can be obtained outside the human body. This significantly increases the convenience of obtaining the information of an implant by the patient or the user, even by the doctor.

### BRIEF DESCRIPTION OF THE DRAWINGS

FIG. 1A is a diagram of the X-ray developable identifying element for use in an implant of Example 1, wherein the body thereof is in the shape of a disc;

FIG. 1B is a diagram of the X-ray developable identifying element for use in an implant of Example 2, wherein the body thereof is in the shape of a sphere;

FIG. 1C is a diagram of the X-ray developable identifying element for use in an implant of Example 3, wherein the body thereof is in the shape of a capsule;

FIG. 2 is a three-dimensional diagram of an implant comprising the X-ray developable identifying element of Example 4;

FIG. 3 is a sectional view diagram of the implant comprising the X-ray developable identifying element of Example 4;

FIG. 4 is a top view diagram of the implant comprising the X-ray developable identifying element of Example 4;

FIG. 5 is a three-dimensional diagram of an implant comprising the X-ray developable identifying element of Example 5;

FIG. 6 is a sectional view diagram of the implant comprising the X-ray developable identifying element of Example 5;

FIG. 7 is a three-dimensional diagram of an implant comprising the X-ray developable identifying element of Example 6;

FIG. 8 is a three-dimensional diagram of an implant comprising the X-ray developable identifying element of Example 7;

FIG. 9 is a three-dimensional diagram of an implant comprising the X-ray developable identifying element of Example 8;

FIG. 10 is a sectional view diagram of the implant comprising the X-ray developable identifying element of Example 8;

### DETAILED DESCRIPTION OF THE PREFERRED EMBODIMENTS

The technical means adopted for achieving the predetermined purpose of the present invention is further explained through the accompanied drawings, and the following preparation examples and examples of the present invention. It should be understood that the descriptions proposed herein are just preferable examples for the purpose of illustrations only, not intended to limit the scope of the disclosure. Various modifications and variations could be made in order to practice or apply the instant disclosure without departing from the spirit and scope of the disclosure.

### Example 1: X-Ray Developable Identifying Element

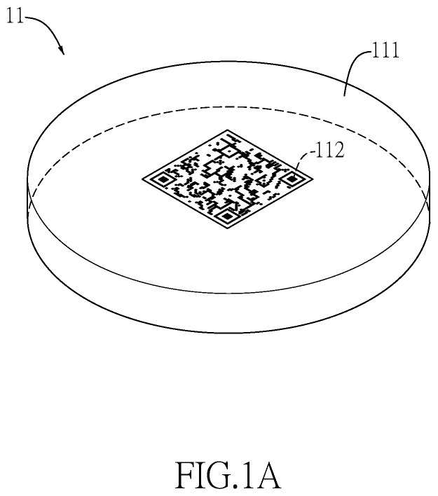

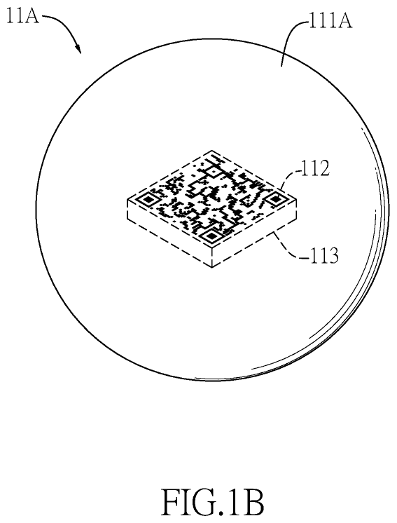

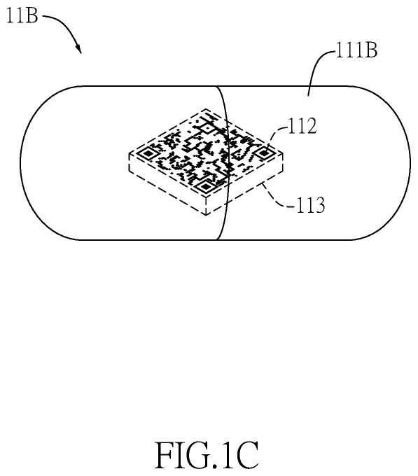

An example of an X-ray developable identifying element for use in an implant 11 in accordance with the present invention is illustrated in FIG. 1. The X-ray developable identifying element for use in an implant 11 comprises a body 111 and an identifying pattern 112, wherein the body 111 is in the shape of a disc, the identifying pattern 112 is formed on the body 111 by printing, wherein the material of the identifying pattern 112 is an X-Ray developer, which is an X-Ray developable material; and the material of the body 111 is silicone, which is an X-Ray undevelopable material.

Since the identifying element 11 contains an identifying pattern 112, which can develop after X-ray radiation, once the identifying element 11 is applied in an implant implanted in a user's body, the X-ray film can display the image of identifying pattern 112, which further provides the information of the brand and product model of the implant, after X-ray detection. Therefore, the patient or the user of the implant can obtain the information of the implant in their bodies. In the present example, the identifying pattern 112 is a quick-response code, which can provide doctors, patients or users with detailed product information of lot number, material or place of origin, after scanning by a scanner.

### Example 2: X-Ray Developable Identifying Element

FIG. 1B illustrates the other example of an X-ray developable identifying element 11A in accordance with the present invention. The X-ray developable identifying element 11A comprises a body 111A and an identifying pattern 112, wherein the body 111A is in the shape of a sphere. Specifically, a substrate 113 is embedded in the body 111A and an identifying pattern 112 is formed on the substrate 113 by printing, wherein the material of the identifying pattern 112 is an X-Ray developer, which is an X-Ray developable material; and the material of the body 111 and of the substrate 113 is silicone, which is an X-Ray undevelopable material.

### Example 3 X-Ray Developable Identifying Element

FIG. 1C illustrates another example of an X-ray developable identifying element 11B in accordance with the present invention. The X-ray developable identifying element 11B of Example 3 is similar to Example 2. The difference between Example 2 and the present Example is that the body 111B of the identifying element 11B in the present example is in the shape of a capsule. Specifically, a substrate 113 is also embedded in the body 111B and an identifying pattern 112 is formed on the substrate 113.

### Example 4: Implant

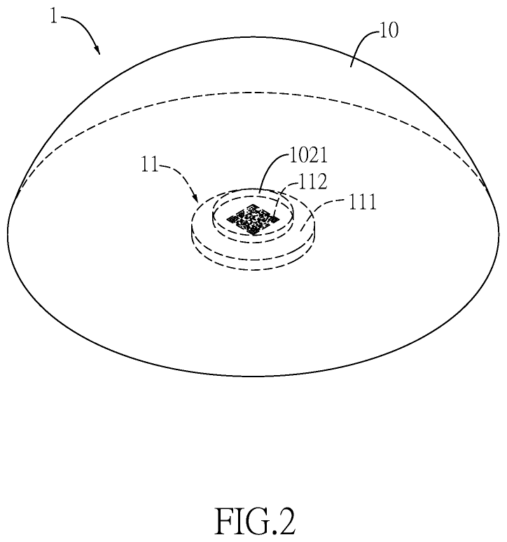

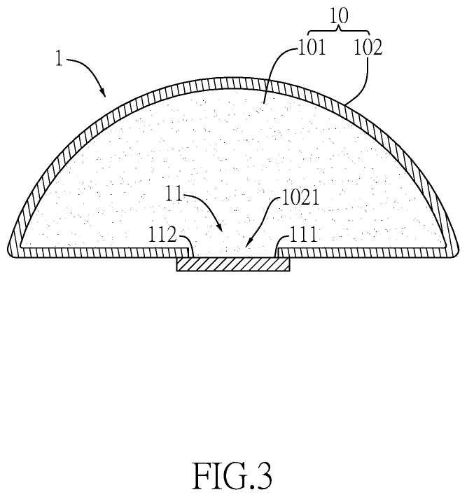

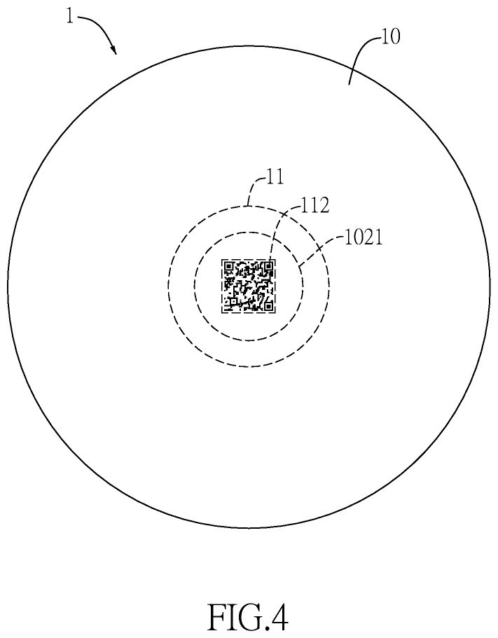

FIGS. 2 to 4 illustrate an implant 1 in accordance with the present invention. The implant 1 comprises a carrier 10 and an X-ray developable identifying element 11 as stated in Example 1. The X-ray developable identifying element 11 is disposed on the carrier 10. Specifically, the implant 1 of the present example is a breast implant and the carrier 10 is composed of a filler 101 and an out-layer 102, wherein a surface of the filler 101 is covered by the out-layer 102, and the out-layer 102 has a hole 1021 for filling the filler 101 into the out-layer 102. Additionally, the X-ray developable identifying element 11 is attached on the hole 1021 to seal the hole 1021, and the identifying pattern 112 on the X-ray developable identifying element 11 faces to the filler 101. As the material of the body 111 of the X-ray developable identifying element 11 of Example 1 for sealing the hole 1021 and the material of the out-layer 102 are both silicone, which is a bio-compatible material, the body rejection hardly occurs when the implant 1 is implanted in a human body, since the body-contacting parts of the implant 1 are made of a bio-compatible material. Furthermore, not only the material of the out-layer 102 is an X-ray undevelopable material of silicone, but also the material of the filler is an X-ray undevelopable material of a silicone gel. Since the material of the carrier 10 and the material of the body 111 of the X-ray developable identifying element 11 are both X-ray undevelopable materials and only the material of the identifying pattern 112 is an X-ray developable material, when the implant of the present example is applied in a patient's or the user's body, there will be only the image of identifying pattern 112 presenting on the X-ray film after X-ray detection. In the present example, the identifying pattern 112 is a quick-response code, which can provide doctors, patients or users with detailed product information of lot number, material or place of origin, after scanning by a scanner. The advantage of the present invention is that the information of the implant in the patient's or user's body can be obtained outside the human body. This significantly increases the convenience of obtaining the information of an implanted that has been implanted in a human body.

In other embodiments, the conventional method of manufacturing an implant can be adopted. That is, attaching an X-ray developable identifying element 11 after filling the silicone to obtain an implant 1, wherein the identifying pattern 112 on the X-ray developable identifying element 11 faces to the silicone filler.

### Example 5: Implant

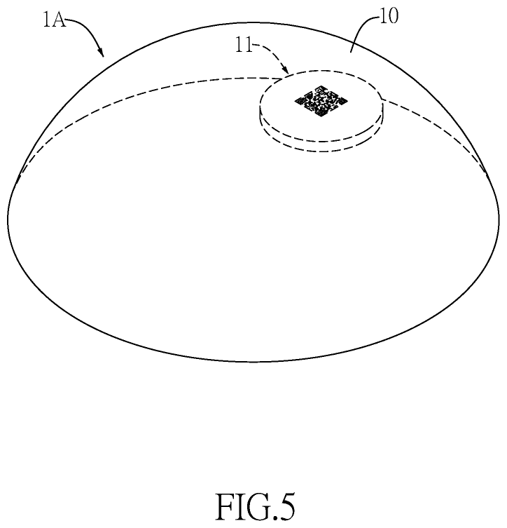

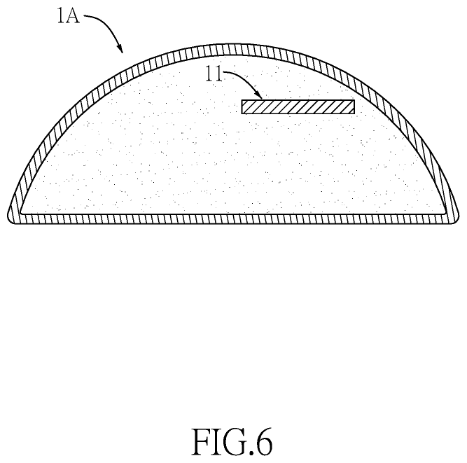

FIG. 5 and FIG. 6 illustrate the other example of an implant 1A in accordance with the present invention. The implant 1A also comprises the X-ray developable identifying element 11 as stated in Example 1. The implant of Example 5 is similar to Example 4, which is also a breast implant. The difference between Example 4 and Example 5 is that the X-ray developable identifying element 11 of the present example is not disposed on the carrier 10, but is embedded in the carrier 10 instead, and the X-ray developable identifying element 11 is movably disposed in the carrier 10. For example, the X-ray developable identifying element 11 is floating or moving in the carrier 10.

### Example 6 Implant

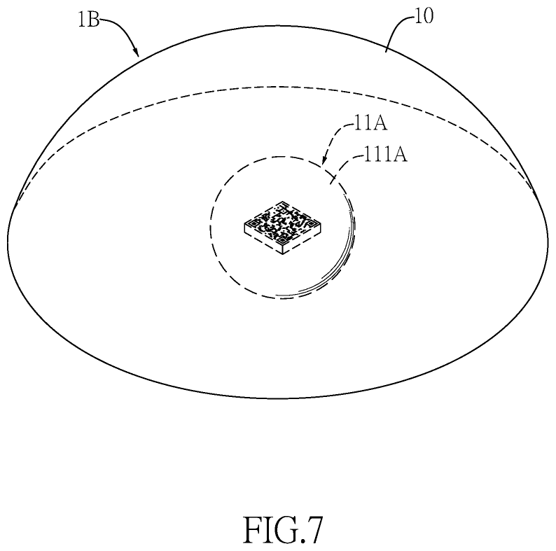

FIG. 7 illustrates the other example of an implant 1B in accordance with the present invention. The implant 1B comprises the X-ray developable identifying element 11A as stated in Example 2. Basically, the implant of Example 6 is similar to Example 5. The difference between Example 5 and Example 6 is the shape of the X-ray developable identifying element 11A and body 111A. The body 111A of the present example is in the shape of a sphere. In addition, the X-ray developable identifying element 11A is not disposed movably, such as floating or moving, in the carrier 10; instead, the X-ray developable identifying element 11A is secured in the carrier 10.

### Example 7 Implant

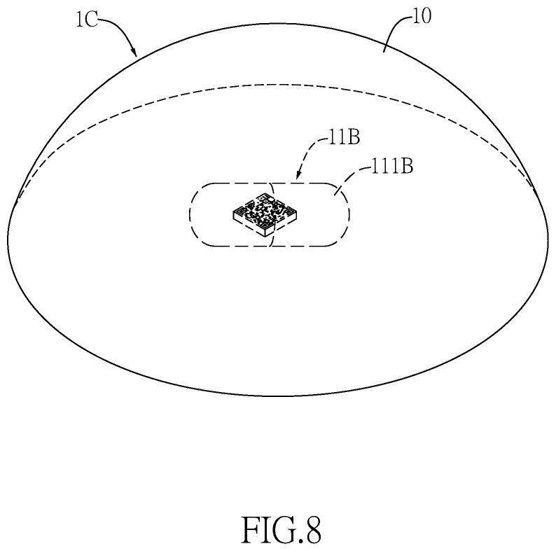

FIG. 8 illustrates the other example of an implant 1C in accordance with the present invention. The implant 1C comprises the X-ray developable identifying element 11B as stated in Example 3. The implant of Example 7 is similar to Example 6. The difference between Example 7 and Example 6 is the shape of the body 111B of the X-ray developable identifying element 11B. The body 111B of the present example is in the shape of a capsule.

### Example 8 Implant

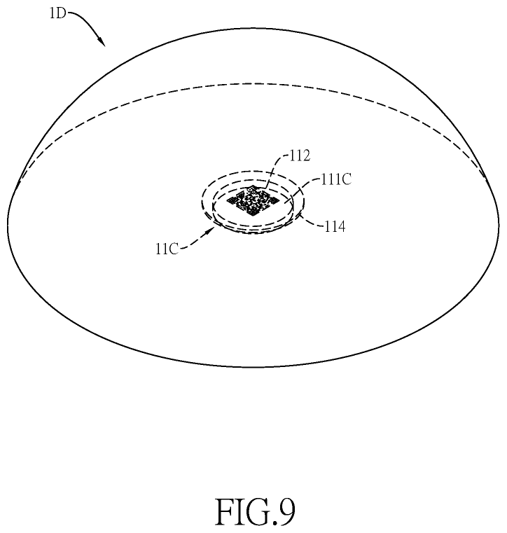

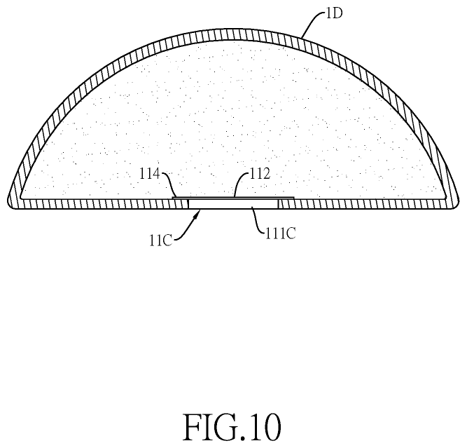

FIG. 9 and FIG. 10 illustrate the other example of an implant 1D in accordance with the present invention. The implant of Example 8 is similar to Example 4. The difference between Example 8 and Example 4 is an X-ray developable identifying element 11C. An identifying pattern 112 is formed on the body 111C of the X-ray developable identifying element 11C of the Example 8 by printing, and a flange 114 is formed outward from the upper end of the body 111C, wherein the body 111C is attached to the implant 11D with the flange 114.

To sum up, the X-ray developable identifying element of the present invention and the implant comprising the same have the identifying pattern, which can develop after X-ray radiation, therefore, the product information of the implant implanted in the human body, such as the brand, lot number, material, place of origin, can be obtained after X-ray radiation and scanning the identifying pattern on the X-ray film by a scanner. Thus, the patient or the user of the implant does not need to go back to the original medical institution for obtaining the information of the implant which has been implanted in the body. This significantly increases the convenience of obtaining the information of an implanted implant by the patient or the user, even by the doctor.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An X-ray developable identifying element for use in an implant, comprising:
a body and an identifying pattern;
wherein the identifying pattern is formed on the body or embedded in the body, and a material of the identifying pattern is an X-ray developable material.

2. The X-ray developable identifying element for use in an implant according to claim 1, wherein the X-Ray developable material is an X-Ray developer or a metal.

3. The X-ray developable identifying element for use in an implant according to claim 1, wherein the material of the body is an X-Ray undevelopable material.

4. The X-ray developable identifying element for use in an implant according to claim 3, wherein the material of the X-Ray undevelopable material is a non-metallic material.

5. The X-ray developable identifying element for use in an implant according to claim 1, wherein a flange is formed outward from an upper end of the body.

6. The X-ray developable identifying element for use in an implant according to claim 1, wherein the identifying pattern comprises a two-dimensional barcode.

7. The X-ray developable identifying element for use in an implant according to claim 6, wherein the two-dimensional barcode is a quick-response code.

8. An implant, comprising:
a carrier, and
the X-ray developable identifying element for use in an implant according to claim 1;
wherein the X-ray developable identifying element is disposed on or embedded in the carrier.

9. An implant, comprising:
a carrier, and
the X-ray developable identifying element for use in an implant according to claim 2;
wherein the X-ray developable identifying element is disposed on or embedded in the carrier.

10. An implant, comprising:
a carrier, and
the X-ray developable identifying element for use in an implant according to claim 3;
wherein the X-ray developable identifying element is disposed on or embedded in the carrier.

11. An implant, comprising:
a carrier, and
the X-ray developable identifying element for use in an implant according to claim 4;
wherein the X-ray developable identifying element is disposed on or embedded in the carrier.

12. An implant, comprising:
a carrier, and
the X-ray developable identifying element for use in an implant according to claim 5;
wherein the X-ray developable identifying element is disposed on or embedded in the carrier.

13. The implant according to claim 8, wherein the carrier comprises a filler and an out-layer; wherein a surface of the filler is covered by the out-layer; when the X-Ray developable identifying element is disposed on the carrier, the X-Ray developable identifying element is disposed on the out-layer; and when the X-Ray developable identifying element is embedded in the carrier, the X-ray developable identifying element is embedded in the filler.

14. The implant according to claim 9, wherein the carrier comprises a filler and an out-layer; wherein a surface of the filler is covered by the out-layer; when the X-Ray developable identifying element is disposed on the carrier, the X-Ray developable identifying element is disposed on the out-layer; and when the X-Ray developable identifying element is embedded in the carrier, the X-ray developable identifying element is embedded in the filler.

15. The implant according to claim 10, wherein the carrier comprises a filler and an out-layer; wherein a surface of the filler is covered by the out-layer; when the X-Ray developable identifying element is disposed on the carrier, the X-Ray developable identifying element is disposed on the out-layer; and when the X-Ray developable identifying element is embedded in the carrier, the X-ray developable identifying element is embedded in the filler.

16. The implant according to claim 11, wherein the carrier comprises a filler and an out-layer, wherein a surface of the filler is covered by the out-layer; when the X-Ray developable identifying element is disposed on the carrier, the X-Ray developable identifying element is disposed on the out-layer; and when the X-Ray developable identifying element is embedded in the carrier, the X-ray developable identifying element is embedded in the filler.

17. The implant according to claim 13, wherein the material of the filler and of the out-layer is an X-ray undevelopable material.

18. The implant according to claim 17, wherein the material of the X-Ray undevelopable material is a non-metallic material.

19. The implant according to claim 18, wherein the non-metallic material is ceramic, glass, polyurethane or silicone.

20. The implant according to claim 8, wherein the implant is a breast implant, an artificial nose implant, an artificial bone, an artificial blood vessel, an intraocular lens or a dental bone graft.
